(19) United States
(12) Patent Application Publication
(10) Pub. No.: US 2003/0012696 A1
(43) Pub. Date: Jan. 16, 2003

(54) Continous analyzer of volatile organic compounds, device and method for continuously assessing the quality of inside ambient air and use of said device for monitoring a ventilation installation

(76) Inventor: Bernard Millancourt, Saint Germaine-eu-Laye (FR)

Correspondence Address: PEARNE & GORDON LLP, 526 SUPERIOR AVENUE EAST, SUITE 1200, CLEVELAND, OH, 44114-1484, US

(21) Appl. No.: 10/220,104

(22) PCT Filed: Mar. 8, 2001
(86) PCT No.: PCT/FR01/00695

(51) Int. Cl. G01N 33/00

(52) U.S. Cl. 422/83; 436/151; 436/116; 436/133

(57) ABSTRACT

The present invention concerns a continuous analyser of volatile organic compounds (10) comprising a circuit (18) for the sequential processing of air such that the air is drawn in by a pump (17) through a filter (11) and scanned by a first sensor (15) for CO/VOC and the second sensor (16) for H₂O, either directly along a first pathway, or after passing through a cartridge (12) for retaining organic species along a second pathway; the switch over from one to the other of these two pathways being assured by an electric valve (13) controlled by a sequencer (14).

The present invention also concerns a device and a method for continuously evaluating the quality of interior ambient air.

### DESCRIPTION

1. Technical Field

The present invention concerns a continuous analyser of volatile organic compounds (VOCs), a device and method for continuously evaluating the quality of interior ambient air and a use of this device for controlling a ventilation unit.

2. State of the Prior Art

Different parameters may be used to characterise the quality of interior ambient air, and particularly the concentrations in H₂O, CO₂, CO, NOₓ, and VOC. If each of these compounds is successively analysed, one has:

H₂O: The hygrometry and the gravimetric concentration in water are recognised comfort factors. They are also, like CO₂, indicators of human presence but a lot less precise, given the great variability in the natural humidity levels in air, and the low emissions linked to human presence in comparison to the high levels of ambient air.

CO₂: Carbon dioxide is not really considered as a pollutant, but it is an excellent indicator of human presence in service sector premises. It is also a good indicator of poor ventilation in residential premises, in particular when cooking equipment or extra heating devices are being used.

CO: Carbon monoxide is a pollutant whose presence in service sector premises is essentially due to the intake of polluted air from the exterior, faulty combustion or even tobacco smoke. In residential premises it is responsible for a considerable number of mortal accidents each year due to faulty combustion devices or devices not connected to smoke exhaust ducts.

NOₓ: Nitrogen oxides may be represented by the dioxide NO₂, which is the most noxious and the only oxide concerned by external ambient air regulations. In service sector premises, the presence of NOₓ is essentially due to the intake of polluted air from the exterior.

VOC: The term Volatile Organic Compound covers a considerable number of compounds whose noxiousness is very variable. Among these, formaldehyde (HCHO) is chosen as the indicator; it is a product of the degradation of materials, frequently emitted in the interior of rooms, irritant to the mucous membranes and whose long term toxicity is now recognised.

These different compounds may be used to establish an air quality “index”.

However, using specific analysers with high metrological performance is out of the question, mainly for cost reasons. In fact, a semi-quantitative determination with good reliability is acceptable.

A first aim of the invention is therefore a continuous analyser of volatile compounds. A further aim is a device and a method for continuously evaluating the quality of interior ambient air, which is not very bulky, easy to use and maintain, of reasonable cost, and capable of rendering an air quality index determined from pollutant levels and their relative noxiousness; enabling the five compounds defined above to be quantified in a reliable and selective manner, in real time, using commercially available micro-sensors.

### DESCRIPTION OF THE INVENTION

The present invention concerns a continuous analyser of volatile organic compounds, characterised in that it comprises:

a measuring module comprising a first CO/VOC sensor and a second H₂O sensor,

a sequential processing circuit for air comprising:

a filter

a cartridge for the selective retention of volatile organic compounds arranged on a first pathway in parallel with a second direct pathway

an electric valve controlled by a sequencer, which assures the first pathway—second pathway commutation

a pump located downstream of the sensors in such a way that the air to be analysed is drawn in through a filter and is transferred towards the CO/VOC and H₂O sensors either directly, or after passing through the cartridge.

a circuit for processing the signals coming from the sensors and the sequencer, enabling the following three parameters to be obtained:

the water content in the air

the CO content in the air, on a sample with the VOCs removed

the VOC content, by calculating the difference of the signals obtained with the help of the CO/COV sensor when the air to be analysed is transferred towards this sensor, either along the first pathway or along the second pathway.

The present invention also concerns a device for continuously analysing the quality of interior ambient air comprising this type of continuous analyser of volatile compounds, in which the measuring module also comprises sensitive elements of sensors for NO₂ and CO₂, and in which the sequential processing circuit for the air drawn in by the pump through the dust filter initially scans the third sensor for NO₂ and the fourth sensor for CO₂, before being transferred towards the first sensor for H₂O and the second sensor for CO/VOC along the first or second pathway.

Advantageously, the first, the second and the third sensors are metal oxide chemical micro-sensors. The pump is a membrane pump.

The present invention also concerns a method for continuously analysing the quality of interior ambient air, implementing the aforementioned device, and which comprises the following stages:

the calibration curve for each of the sensors for measuring different compounds: H₂O, CO/VOC, NO₂ and CO₂ is determined.

the influence of the majority interfering compounds is corrected by calculation.

the output signal from each sensor is transposed into measured compound concentration, while taking account of its calibration curve.

a quality index for each compound measured is determined by referring to an evaluation grid that gives an index value for each compound as a function of different thresholds limits for the concentrations of compounds, in reference to health data.

an overall index of the quality of the air is obtained as a function of the different compound indexes obtained.

The preceding device may be used advantageously for controlling a ventilation unit.

### BRIEF DESCRIPTION OF THE DRAWINGS

FIG. 1 illustrates the device of the invention.

FIG. 2 illustrates the response curve for sensor 20.

FIG. 3 illustrates the calibration curve for sensor 16.

FIG. 4 illustrates a measuring sequence.

FIG. 5 illustrates the exploitation of the output signals from sensors 15 and 16.

### DETAILED DESCRIPTION OF SPECIFIC EMBODIMENTS

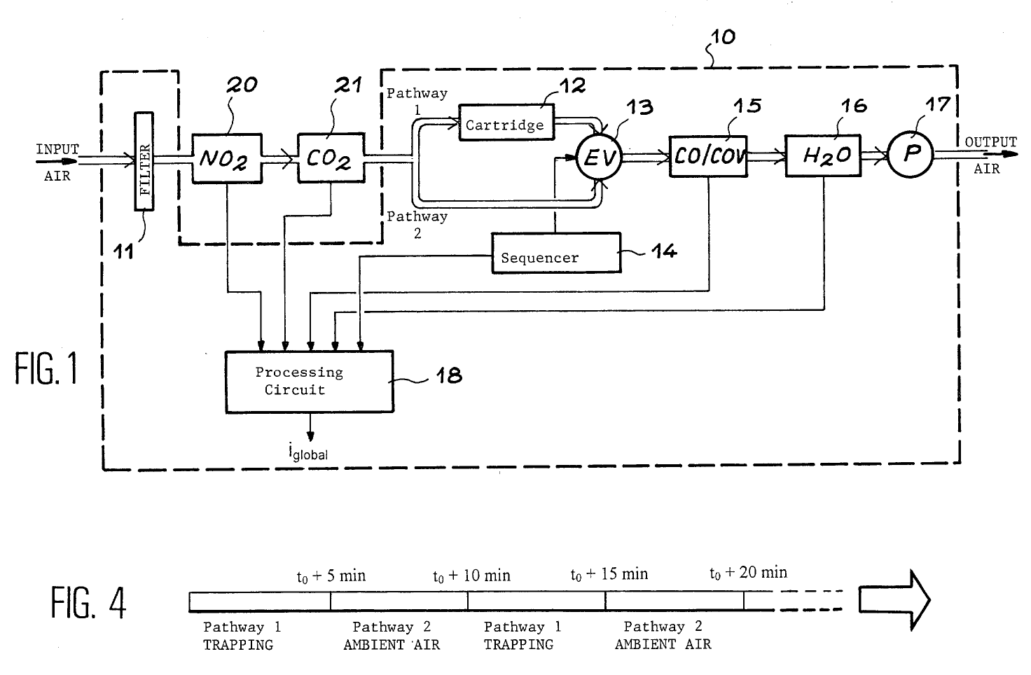

As illustrated in FIG. 1, the continuous analyser of Volatile Organic Compounds 10 successively comprises:

processing circuit for air comprising:

a filter 11, which may be a coarse dust filter

a cartridge 12 for the selective retention of volatile organic compounds arranged on a pathway 2 in parallel with a direct pathway 1.

an electric valve 13 controlled by a sequencer 14, which assures the first pathway—second pathway commutation

a first sensor for CO/VOC 15 and a second sensor for H₂O 16.

a pump 17, which may be a membrane pump.

a circuit 18 for processing the signals coming from the sensors 15 and 16 and the sequencer 14.

The air to be analysed is drawn in by the pump 17 through the filter 11 and is transferred to the CO/VOC 15 and H₂O sensors, either directly, or after going through the cartridge 12; the sequential commutation being assured by the electric valve 13.

The pump 17, which enables the air to be sampled, is placed downstream of the analysis circuit in such a way as to avoid any contamination or retention of species.

The analyser of the invention therefore comprises two main parts:

a measuring module, which comprises the sensitive elements of the sensors CO/VOC 15 and H₂O sensors, supply and measuring circuits and a sequential processing circuit of the sampled air.

a module 18 for processing and exploiting the signals.

The device for continuously evaluating the quality of interior ambient air according to the invention, illustrated in FIG. 1, comprises all of the elements of the analyser 10 of the invention, as defined here above. It moreover comprises a third sensor for NO₂ 20 and a fourth sensor for CO₂ 21 arranged between the filter 11and the pathways 1 and 2.

The method for continuously evaluating the quality of interior ambient air according to the invention, implementing the device defined here above, comprises the following stages:

the calibration curve for each of the sensors 15, 16, 20 and 21 for measuring different compounds: H₂O, CO/VOC, NO₂ and CO₂ is determined.

the influence of the majority interfering compounds is corrected by calculation.

the output signal from each sensor is transposed into measured compound concentration, while taking account of its calibration curve.

a quality index for each compound measured is determined by referring to an evaluation grid, such as that illustrated by way of example in Table 3, which gives an index value for each compound as a function of different thresholds limits for the concentrations of compounds, referring to health data.

an overall index i_{global} of the quality of the air is obtained as a function of the different compound indexes obtained thereof.

### EXAMPLE OF AN EMBODIMENT

In an example of an embodiment, the sensors 15, 16 and 20 for CO/VOC, H₂O and NO₂ used are commercially available metal oxide chemical micro-sensors. This type of sensor is made up of a sensitive semi-conductor element, usually based on tin oxide SnO₂, heated to its optimal operating temperature by a heating element, and whose electrical characteristics vary as a function of the presence in the ambient air of gaseous compounds. The sensitive element is the focus point for absorption—desorption and oxidation—reduction phenomena for which the equilibria are determined principally by the temperature. The electronics of such a sensor are very simple.

The sensor 21 for CO₂ is an infrared (IR) sensor. In fact, CO₂ has the property of absorbing infrared radiation with an absorption maximum between 4000 nm and 4400 nm. For a given geometry of the measuring cell, the radiation absorption is directly linked to the concentration in CO₂ (Beer—Lambert law). This sensor 21 could also be a chemical micro-sensor.

Among the different types of possible sensors, we have retained by way of example the following sensors 15, 16, 20, 21:

CO/VOC : FIGARO TGS 2620 sensor

H₂O : FIGARO TGS 2180 sensor

NO₂ : FIGARO TGS 2105 sensor

CO₂ : SAUTER IR sensor/type EGQ 220 F001

The sensitive elements of sensors 20 and 21 for NO₂ and CO₂ may be arranged on a support and be exposed directly to the ambient air sampled via the membrane pump.

The sensor 21 for CO₂ is integral with its electronics and is used as provided by the supplier after having been removed from its protective casing for reasons of bulk.

The sensors 15 and 16 for CO/VOC and H₂O are arranged under a cover that makes it possible to scan alternately, either directly by the ambient air, or after passing through the cartridge 12.

The selective retention cartridge 12 for volatile organic compounds may be obtained by using potassium permanganate.

In fact, aldehydes, ketones and alcohols react with potassium permanganate and are fixed by oxidation, in a quantitative manner; benzenic compounds are retained, a priori, by absorption. The flow of air to be purged must be low in order to ensure sufficient contact time for complete trapping, and in practice a flow of 0.3 l/min and a cartridge of 200 mm may be used. A lower flow rate makes it possible to reduce the dimensions of the cartridge without affecting its autonomy. Activated aluminium oxide (alumina Al₂O₃) in microporous beads of 2 mm to 5 mm diameter is used as a support for the active compound comprising potassium permanganate (KMnO₄). In order to obtain a preparation of 100 g, one very simply achieves the impregnation by immersing 100 g of alumina in an acidified aqueous solution (H₂SO₄ 10⁻²N) at 60 g/l of potassium permanganate. After spin drying, the alumina beads are dried at 60-70° C. for around 4 hours and conserved shielded from air, this treatment making it possible to obtain an alumina containing 5% by weight of KMnO₄. 100 g of this preparation enables 6 cartridges of 200 mm/diameter 20 mm to be filled.

The pump 17 may be a WISA type membrane pump used in gas analysers, separated from the sensors for reasons of bulk; but it may also be a smaller pump that can easily be fitted into the measurement box.

The duration of the cycle of the control signal from electric valve 13, supplied by the sequencer 14, may be chosen between 30 seconds and three hours, for example 5 minutes.

The circuit 18 for processing the output signals delivered by the sensors 15, 16, 20, 21 is achieved using an AOIP 70 acquisition unit with its pathways connected to a PC type computer; the mathematical processing of the signals is carried out using EXCEL type software. It is also possible to use microprocessors integrated in the device of the invention.

In this example of an embodiment, the following measurements were made.

Measurement of Nitrogen Dioxide (NO₂)

The measurement was carried out by exposing the sensor 20 for NO₂ to the flow of sampled air.

As illustrated in FIG. 2, the sensor 20 offered a response to the nitrogen dioxide within a relatively narrow concentration field (0 to 200 ppb), but appropriate to the concentrations encountered in the areas considered, with quite good selectivity, thus allowing the signal to be exploited directly.

The impact of the CO on the measurement of the NO₂, effective for high CO/NO₂ concentration ratios (greater than 100), could be disregarded without leading to significant errors.

Measurement of the Humidity (H₂O)

The measurement of H₂O was carried out using sensor 16, which offers good sensitivity and good selectivity to water; its response is linked to the gravimetric concentration in water (expressed in mass/m³ or in ppm) and not the relative humidity of the air.

CO, CO₂, NOₓ and VOCs do not influence the measurement in the areas concerned by ambient air.

The response of this sensor 16, illustrated in FIG. 3, was used not only for measuring the water content but also to correct the influence of this water content on the response to CO and to VOCs of the sensor 15.

Associated Measurements of CO, H₂O and VOCs

The concentrations of CO, H₂O and VOCs were measured using sensors 15 and 16.

The evaluation of the concentrations of CO and Volatile Organic Compounds (VOCs) was carried out using the multi-pollutant sensor 15, which offers good sensitivity to VOCs but requires a correction for the influence of the water concentrations, carried out using the second sensor 16, specific to water.

In interior spaces, VOCs are present at very low concentrations compared to the levels of potentially interfering compounds such as CO or H₂O, which makes the corrections by purely mathematical route very uncertain. This difficulty is overcome by carrying out a selective trapping of VOCs using the cartridge 12, upstream of sensor 15, and by alternately introducing purged air and the air to be analysed into this sensor 15. The major interfering agents are not trapped, and the differences in the signals makes it possible to obtain, with good sensitivity, the concentration of VOCs.

The ranges concerned are as follows:

H₂O: 5000 to 25000 ppm

CO: 0 to 25 ppm

Total VOC: several tens of ppb to 1 ppm.

By calculating differences in the stabilised signals during each sequence, it is possible to obtain the concentration of the masked compound while at the same time being able to disregard the concentrations of interfering compounds as well as drifts from zero of the sensor.

The two associated sensors 15 and 16 thus make it possible, according to the signal sampling period, to obtain the following three parameters with good accuracy:

the water concentration of the ambient air

the CO concentration of the ambient air on a sample cleared of VOCs

the VOC concentration, by calculating the difference in one signal and another.

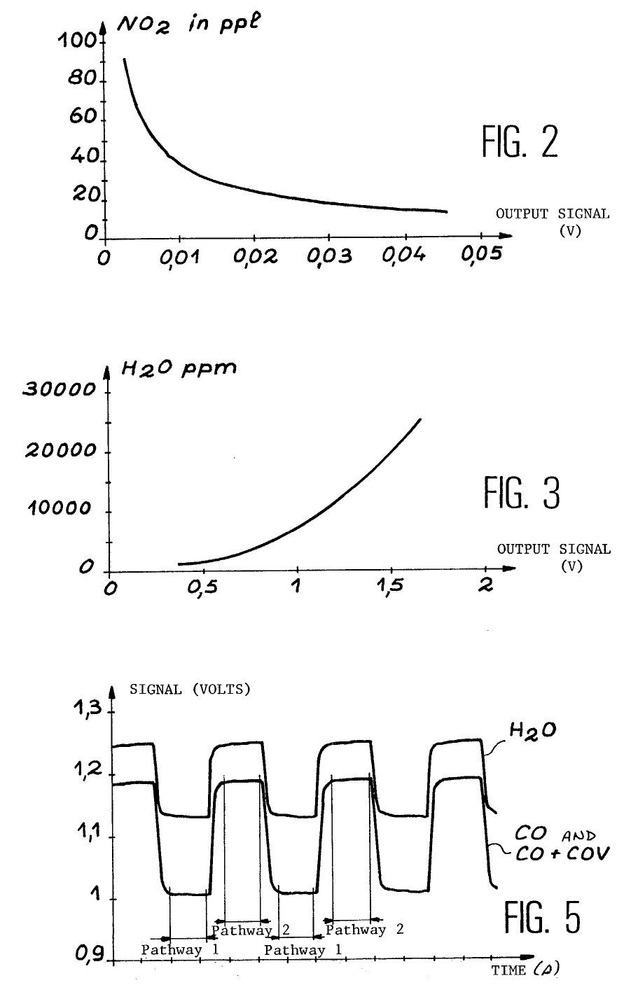

The air sampled using pump 17 was thus introduced into the two sensors 15 and 16, either directly or after passing through the cartridge 12 as illustrated in FIG. 4. 5 minute sequences were chosen.

The raw signals delivered by these sensors 15 and 16 were sampled after stabilisation, in the following manner:

Pathway 1 (ambient air) [symbol] sensor 16 [symbol] measurement of ambient H₂O

Pathway 2 (cartridge) [symbol] sensor 15 [symbol] measurement of ambient CO (freed of trapped VOCs)

(Pathway 1—pathway 2) [symbol] measurement of trapped VOCs.

The signal sampling phases are illustrated in FIG. 5, which shows a typical evolution of signals during a test.

Measurement of CO₂

The measurement was carried out using the sensor 21. This infrared sensor, removed from its protective casing, was integrated without any modification to the device. This sensor has good response linearity in its measuring range (0 to 2000 ppm), and good sensitivity.

To process the output signals from the sensors, the following calibration curves were considered:

Nitrogen Dioxide NO₂/Sensor 20

The calibration curve is of the type:

[NO₂]=a E⁽ⁿ⁾

where: [NO₂] represents the concentration expressed in ppb and E represents the signal from the sensor (in volts).

Gravimetric Concentration in Water (H₂O)/Sensor 16 “Pathway 1”

The equation for the calibration curve for sensor 16 is of the type:

H₂O_{in ppm}=b. (E−E₀)²+c. (E−E₀)+d

Where: E represents the raw voltage delivered by the sensor (in volts), and

E₀ represents the base voltage of the sensor.

Carbon Monoxide (CO)/sensor 15 “Pathway 2”

The equation for the calibration curve for the sensor 15 vis-à-vis CO is a polynomial of the second degree of the type:

CO_{in ppm}=e. [E−E₀−E_{(H2O)}]²+f. [E−E₀−E_{(H2O)}]+g

Where: E represents the raw voltage delivered by the sensor 2620 (in volts),

E₀ represents the base voltage of the sensor 15.

E_{(H2O)} represents the correction for the influence of the water concentration on the sensor 15, from the concentration delivered by the sensor 16.

Formaldehyde and Volatile Organic Compounds Expressed as “Formaldehyde Equivalents”/Sensor 15 “Pathway 1—Pathway 2”

Among the major VOCs in polluted ambient air, the cartridge 12 quantitatively traps the following compounds and families of compounds:

formaldehyde and other aldehydes

ketones (acetone, etc.)

alcohols (methanol, ethanol, etc.)

benzenic compounds.

All of these compounds have a recognised toxicity and the sensor 15 has similar sensitivity to them. Among these pollutants, formaldehyde turns out to be in the majority in interior premises and it is all of these “undesirable” VOCs taken together that is expressed as “formaldehyde equivalents”.

The CO (and alkanes), present in the air at concentrations that can reach several ppm, is not trapped by the cartridge 12.

CO, which is toxic, is measured during the sequence corresponding to pathway 2. The measurement of CO integrates the possible presence of alkanes. If these compounds are present, the measurement is carried out by excess; this constitutes an asset by allowing the device to react to the presence of methane, in the event of a leak of natural gas for example.

For each response level, the averages in pathway 1 and pathway 2 are calculated by eliminating the stabilisation phases (around 1 minute before and after each commutation).

For the sensor 15, one has:

Pathway 2 (trapping) : 0.5×[average (t₀+7 to t 0+9)+average (t₀+17 to t₀+19)] (average of signals from “pathway 2” sequences preceding and following a “pathway 1” sequence).

Pathway 1 (direct passage) : average (t₀+12 to t₀+14)

For the sensor 16, one has:

Pathway 2 (trapping): 0.5×[average (t₀+7 to t₀+9)+average (t₀+17 to t₀+19)] (average of signals from “pathway 2” sequences preceding and following a “pathway 1” sequence).

Pathway 1 (direct passage) average (t₀+12 to t₀+14).

The influence of water concentration variations in the sensor 15 is corrected very simply by assigning the difference in the signals “pathway 1—pathway 2”, measured on sensor 16, a coefficient S representing the ratio of sensitivities respectively of these two sensors to water, in other words the ratio of the slopes of the two response curves in a humidity range going from 5000 to 25000 ppm.

The responses from sensors 15 and 16 (raw differential voltages in volts) in the extreme water content ranges encountered in exterior air are given in Table 1 at the end of the description. The equation for the variation curve (assimilated to a straight line) of this ratio as a function of the water concentration is: Coefficient S=2.10⁻⁵ [H₂O].

The variations in the water concentrations at the level of sensor 15, downstream of cartridge 12, are between 0 and ±6000 ppm; a fixed ratio of 1.67 is thus retained between the raw voltages delivered by the sensors 15 and 16 for a same water content.

The value of 1.67 corresponding to the maximum water content difference is retained preferentially to an average coefficient, since it enables a better correction matching in so far as only high differences have a notable impact on the results. One thus obtains after correction of the response of the water concentration from sensor 15:

VOC (in mg/m³ of HCHO)=K₁×(Δ(V1−V2)₂₆₂₀−1.67×Δ(V1−V2)₂₁₈₀)

Where K₁ is the slope of the response to VOCs of the sensor 15.

If the second term enables the response to water of sensor 15 to be corrected, the difference “pathway 1−pathway 2” of the first term enables the response of this sensor to the CO not trapped by the cartridge 12 to be corrected and to allow any drifts from zero of the sensor 15 over time to be disregarded.

A calibration is carried out by injecting and vaporising known quantities of HCHO in a 37% aqueous solution; table 2 at the end of the description shows values of signals after the treatment described above. The calibration curve is a straight line in a concentration range between 0 and 6 mg/m³.

Carbon Dioxide/Sensor 21

The equation for the calibration curve is of the type:

CO₂ in ppm=a×E+b

in which E represents the signal expressed in volts (1-10 V for 0-2000 ppm).

Establishing an Air Quality Index

Various approaches for establishing such an index may be envisaged; one solution consists in comparing the measured concentration of each of the selected compounds: H₂O, CO, NO₂, HCHO, CO₂ at different thresholds, as in the grid in Table 3.

The concentration levels that could be attained by each of these levels are broken down into 10 classes, established either from regulatory thresholds, if they exist, or from the recommendations of the World Health Organisation for the protection of health and each constituting an elementary index.

The overall index is represented by the highest index of the elementary indices corresponding to each of the selected compounds.

An example of a CO index is as follows: Regulatory limit in working atmospheres: 50 ppm over a period of 8 h.

WHO recommendations:

60 mg/m³ (≈50 ppm) during 30 minutes

30 mg/m³ (≈25 ppm) during 1 hour

10 mg/m³ (≈5 ppm) during 8 hours.

Maximum retained for the index:

20 ppm (23 mg/m³ [symbol] index 10).

In the same way, an index 10 corresponds to:

1 mg/m³ of VOC expressed in HCHO equivalents (0.8 ppm at 20° C.)

200 μg/m³ of NO₂ (109 ppb at 20° C.)

2000 ppm of CO₂ (3667 mg/m³)

TABLE 1

| | TGS 2620 | | |
|---|---|---|---|
| H₂O in ppm | signal | TGS 2180 signal | Ratio |
| 2500 | 0.1938 | 0.1194 | 1.623 |
| 5000 | 0.3750 | 0.2275 | 1.648 |
| 6000 | 0.4440 | 0.2676 | 1.659 |
| 7000 | 0.5110 | 0.3059 | 1.670 |
| 8000 | 0.5760 | 0.3424 | 1.682 |
| 9000 | 0.6390 | 0.3771 | 1.695 |
| 10000 | 0.7000 | 0.4100 | 1.707 |
| 11000 | 0.7590 | 1.4411 | 1.721 |
| 12000 | 0.8160 | 0.4704 | 1.735 |
| 13000 | 0.8710 | 0.4979 | 1.749 |
| 14000 | 0.9240 | 0.5236 | 1.765 |
| 15000 | 0.9750 | 0.5475 | 1.781 |
| 16000 | 1.0240 | 0.5696 | 1.798 |
| 17000 | 1.0710 | 0.5899 | 1.816 |
| 18000 | 1.1160 | 0.6084 | 1.834 |
| 19000 | 1.1590 | 0.6251 | 1.854 |
| 20000 | 1.2000 | 0.6400 | 1.875 |
| 22000 | 1.2760 | 0.6644 | 1.921 |
| 25000 | 1.3750 | 0.6875 | 2.000 |

TABLE 2

| HCHO μg/m³ | Signal in volts |
|---|---|
| 0 | 0.0064 |
| 28.5 | 0.0118 |
| 28.5 | 0.0113 |
| 47.5 | 0.0168 |
| 47.5 | 0.0153 |
| 95 | 0.225 |
| 142.5 | 0.241 |
| 190 | 0.301 |
| 237.5 | 0.359 |
| 237.5 | 0.347 |

TABLE 3

| CO level | HCHO level | | NO₂ level | | CO₂ level | |
|---|---|---|---|---|---|---|
| mg/m³ | μg/m³ | Index | μg/m³ | Index | ppm | Index |
| <1 | <50 | 0 | <20 | 0 | <650 | 0 |
| 1 to 2 | 50 to 75 | 1 | 20 to 40 | 1 | 650 to 800 | 1 |
| 2 to 4 | 75 to 100 | 2 | 40 to 60 | 2 | 800 to 950 | 2 |
| 4 to 8 | 100 to 150 | 3 | 60 to 80 | 3 | 950 to 1100 | 3 |
| 8 to 10 | 150 to 200 | 4 | 80 to 100 | 4 | 1100 to 1250 | 4 |
| 10 to 12 | 200 to 300 | 5 | 100 to | 5 | 1250 to 1400 | 5 |
| 12 to 14 | 300 to 400 | 6 | 120 to | 6 | 1400 to 1550 | 6 |
| 14 to 16 | 400 to 600 | 7 | 140 to | 7 | 1550 to 1700 | 7 |
| 16 to 18 | 600 to 800 | 8 | 160 to | 8 | 1700 to 1850 | 8 |
| 18 to 20 | 800 to 1000 | 9 | 180 to | 9 | 1850 to 2000 | 9 |
| CO > 20 | >1000 | 10 | >200 | 10 | >2000 | 10 |

1. Continuous analyser of volatile organic compounds characterised in that it comprises:
a measuring module comprising a first CO/VOC sensor (15) and a second H₂O sensor (16),
a sequential processing circuit for air comprising:
a filter (11)
a cartridge (12) for the selective retention of volatile organic compounds arranged on a first pathway (12) in parallel with a second direct pathway (11)
an electric valve (13) controlled by a sequencer (14), which assures the first pathway—second pathway commutation
a pump (17) located downstream of the sensors (15, 16) in such a way that the air to be analysed is drawn in through a filter (11) and is transferred towards the CO/VOC (15) and H₂O (16) sensors either directly, or after passing through the cartridge (12)
a circuit (18) for processing the signals coming from the sensors (15, 16) and the sequencer (14), enabling the following three parameters to be obtained:
the water concentration of the air
the CO concentration of the air, on a sample with the VOCs removed
the VOC concentration, by calculating the difference in signals obtained using the CO/VOC sensor (15) when the air to be analysed is transferred towards this sensor either along the first pathway or along the second pathway.

2. Device for continuously analysing the quality of interior ambient air comprising a continuous analyser of volatile compounds according to claim 1, in which the measuring module also comprises sensitive elements of sensors for NO₂ (20) and CO₂ (21), and in which the sequential processing circuit for the air drawn in by the pump through the filter (11) initially scans the third sensor (20) and the fourth sensor (21) before being transferred towards the first sensor (15) and the second sensor (16) along the first or second pathway.

3. Device according to claim 2, in which the first, the second and the third sensors (15, 16, 20) are metal oxide chemical micro-sensors.

4. Device according to claim 2, in which the pump (17) is a membrane pump.

5. Method for continuously analysing the quality of interior ambient air, implementing the device according to any of claims 2 to 4, and which comprises the following stages:
the calibration curve for each of the sensors (15, 16, 20, 21) for measuring different compounds: CO/VOC, H₂O, NO₂ and CO₂ is determined.
the influence of the majority interfering compounds is corrected by calculation.
the output signal from each sensor is transposed into measured compound concentration, while taking account of its calibration curve.
a quality index for each compound measured is determined by referring to an evaluation grid that gives an index value for each compound as a function of different thresholds limits for the concentrations of compounds, referring to health data.
an overall index of the quality of the air is obtained as a function of the different compound indexes obtained thereof.

6. Use of the device according to any of claims 2 to 4 for controlling a ventilation unit.
